(19) United States
(12) Patent Application Publication
(10) Pub. No.: US 2011/0177592 A1
(43) Pub. Date: Jul. 21, 2011

(54) MAGNETIC APPARATUS FOR BLOOD SEPARATION

(76) Inventors: Denise L. Faustman, Boston, MA (US); Douglas E. Burger, Stoughton, MA (US)

(21) Appl. No.: 13/003,435

(22) PCT Filed: Jul. 13, 2009
(86) PCT No.: PCT/US09/50376

§ 371 Date: Apr. 6, 2011

Related U.S. Application Data
(60) Provisional application No. 61/080,083, filed on Jul. 11, 2008.

(51) Int. Cl. C12N 5/02; C12M 1/00

(52) U.S. Cl. 435/325; 435/283.1

(57) ABSTRACT

An apparatus for magnetically separating constituents of blood includes: a first portion having a solid support haying fixedly attached thereon a plurality of ring magnets, each ring magnet defining an interior perimeter enclosing an aperture; and a second portion removably attached to the first portion, wherein the second portion includes a solid substrate having surfaces defining a plurality of second apertures, each second aperture configured to removably hold a vial.

### CLAIM OF PRIORITY

This application claims priority to U.S. Patent Application Ser. No. 61/080,083, filed on Jul. 11, 2008, the entire contents of which are hereby incorporated by reference.

### TECHNICAL FIELD

This invention relates to magnetic separations of liquids, e.g., for separation of blood constituents such as white blood cells.

### BACKGROUND

Blood is comprised of cells and plasma. The cellular portion includes several kinds of cells, including red blood cells (RBCs) and white blood cells (WBCs). The number of RBCs exceeds the number of WBCs by a factor of more than 1,000:1. In many clinical settings, it is useful to separate viable WBCs or a subpopulation of WBCs from RBCs. For example, in the HIV/AIDS field it would be of benefit to know if specific T cell subpopulations are present in the blood and are capable of eliciting an immune response to the virus. In the vaccination clinical trial arena, it is desirable to know if an immunization induces the formation of disease fighting cells. In the field of autoimmunity, it is useful to both isolate pathogenic T cells and perform functional studies on these cells.

Magnetic particles can be used to separate subpopulations of blood cells. Typically, subpopulations are separated either by positive selection using magnetically labeled antibodies that bind to cell surface markers of the desired blood cells (e.g., WBCs or a cellular subpopulation of WBCs, e.g., T cells, B cells, NK cells, monocytes, dendritic cells, granulocytes, or leukocytes), or by negative selection using magnetically labeled antibodies that bind to cell surface markers of RBCs or other blood cells to be removed. Specific methods for magnetic separation of WBCs are described, for example, in U.S. Pat. Nos. 4,910,148; 5,411,863; 6,417,011; and 6,576,428. Kits for performing magnetic separations are commercially available, e.g., from Dynal Biotech (Oslo, Norway) and Miltenyi Biotech (Bergisch Gladbach, Germany), StemCell Technologies (British Columbia, Canada), BD Biosciences (Franklin Lakes, N.J.), Polysciences (Warrington, Pa.), and Bangs Laboratories (Fishers, Ind.).

Nevertheless, there continues to be an unmet need for automation of whole blood separation of WBCs with reproducibly high viability, yield, and purity.

### SUMMARY

The present application provides apparatuses and associated methods useful for automated magnetic separations.

An apparatus includes a first portion that includes a solid support having fixedly attached thereon a plurality of ring magnets, each ring magnet defining an interior perimeter enclosing an aperture. In some embodiments, each ring magnet is configured to receive within its aperture a vial. In some embodiments, each ring magnet is seated within a depression on the surface of the solid support. In some embodiments, the solid support includes an indentation associated with each ring magnet defining an aperture contiguous with the aperture of the ring magnet such that the two apertures are configured to receive within the combined aperture a vial. In some embodiments, the solid support is composed of a non-magnetic or non-magnetizable material, e.g., a non-ferrous metal, plastic, or polymer. In some embodiments, the plurality of ring magnets are evenly spaced in a rectangular array consisting of rows and columns. In some embodiments, each of the plurality of ring magnets is axially magnetized. In some embodiments, the magnetic axis of each of the plurality of ring magnets is inverted relative to its immediate neighbor in its row or column.

In some aspects, the apparatus also includes a second portion that includes a solid substrate having surfaces defining a plurality of apertures, each aperture configured to receive a vial. In some embodiments, the vials are integrated within the structure of the solid substrate. In some embodiments, the apertures are formed by a series of walls forming tubular apertures (e.g., cylindrical apertures or conical apertures). In some embodiments, the surfaces defining the plurality of apertures have within their interior diameter one or more means to removably hold a vial in place, e.g., a vial holder, such as one or more clamps, o-rings, lips, baffles, or ridges. In some embodiments, one or more o-rings are held within one or more grooves on the interior diameter of the surfaces defining the plurality of apertures. In some embodiments, the o-rings are composed of a flexible material, e.g., silicone, viton, ethylene propylene diene M-class rubber, buna-N, rubber, or neoprene. In some embodiments, the substrate includes means for removably holding a vial. In some embodiments, the second portion is configured such that the vials are aligned with the apertures defined by the ring magnets of the first portion. In some embodiments, the second portion has a lower surface configured to receive the upper surface of the first portion. In some embodiments, the second portion also includes a cover or lid, e.g., to isolate and/or maintain the sterility of a sample held within a vial. In some embodiments, one or both of the second portion and cover or lid includes a means, e.g., a handle or groove, for facilitating handling by a robotic arm of an automated laboratory system.

Processes for isolating magnetic particles (e.g., for separating constituents of a liquid sample such as blood) include providing a vial containing a solution that includes magnetic particles; placing the vial within the magnetic field of an apparatus that includes a first portion that includes a solid support having fixedly attached thereon a plurality of ring magnets, such that the particles are held within the vial by the magnetic field of one or more of the ring magnets; and removing the solution from the vial, such that the magnetic particles remain isolated in the vial. In some embodiments, the processes include washing the particles, wherein washing the particles includes removing the vial from the magnetic field, adding a second solution to the vial such that the magnetic particles are suspended within the second solution, placing the vial within the magnetic field of the apparatus such that the particles are held within the vial by the magnetic field of one or more of the ring magnets, and removing the solution from the vial. In some embodiments, the vial is placed within an aperture defined by the interior perimeter of one of the plurality of ring magnets. In some embodiments, the apparatus also includes a second portion that includes a solid substrate having a surface defining an aperture configured to receive the vial. In some embodiments, the surface of the second portion defining the plurality of apertures have within their interior diameter one or more means to removably hold the vial in place and the vial is placed within the magnetic field of the one or more of the ring magnets by moving the second portion of the apparatus vertically with respect to the first portion of the apparatus.

A process for isolating white blood cells from two or more samples includes obtaining two or more samples comprising whole blood or a blood fraction comprising white blood cells; apportioning the two or more samples in an array of vials having a vertical and horizontal component such that each of the two or more samples is divided among two or more vials distributed horizontally, wherein a liquid handling pipettor comprises a set of pipettes arranged vertically; contacting the two or more samples with magnetic particles that bind specifically to white blood cells or a subset of white blood cells using the liquid handling pipettor to provide white blood cell/magnetic particle complexes; and isolating the white blood cell/magnetic particle complexes by subjecting the white blood cell/magnetic particle complexes to a magnetic field, e.g., a magnetic field of an apparatus described herein.

A computer-readable medium includes software encoded therein for causing a robot to isolate magnetic particles. The software can include instructions for causing a robot to retrieve a vial containing a solution having magnetic particles; place the vial within a magnetic field created by a ring magnet, such that the particles are held within the vial by the magnetic field created by the ring magnet; and remove the solution from the vial, such that the magnetic particles remain isolated in the vial. The software can further include instructions for causing the robot to remove the vial from the magnetic field; add a second solution to the vial such that the magnetic particles are suspended within the second solution; place the vial within the magnetic field created by the ring magnet, such that the particles are held within the vial by the magnetic field created by the ring magnet; and remove the solution from the vial.

Advantages of the apparatuses and methods include facilitation of automation for improved yield, improved viability, improved purity (e.g., reduction of contaminating red blood cells), and reduced variation as compared to methods performed by hand. The apparatuses and methods also provide for faster cell isolations, as compared to methods performed by hand, with less human involvement.

A ring magnet that is axially magnetized is one for which a line joining the N and S poles of the magnet crosses a plane defined by the aperture of the ring magnet.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control.

In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an exploded view of a magnetic apparatus.

FIG. 2 is a cross-sectional view of an assembled magnetic apparatus.

FIG. 3 is a cross-sectional view of the second portion of the apparatus.

FIG. 4 is a flowchart showing an exemplary set of steps for a cell preparation. These steps can be performed by an automated laboratory system.

FIG. 5 is a representation of a microplate with twenty-four wells, with three samples allotted in two sets of consecutive columns each.

FIG. 6 is a representation of a microplate with twenty-four wells, with four samples allotted each in one row.

FIG. 7 is a representation of an array of axially magnetized ring magnets with faces having polarities arbitrarily designated north (N) and south (S).

FIG. 8A is a representation of an apparatus with twenty-four sample vials and fifteen post magnets, each indicated with a sample magnetic field axis.

FIG. 8B is a representation of an apparatus with twenty-four sample vials and one ring magnet per vessel.

FIGS. 8C and 8D are representations of sample vials showing magnetic beads (arrowheads) separated using a post magnet apparatus (8C) or a ring magnet apparatus (8D).

FIGS. 8E and 8F are scatter plots showing forward and side scatter for CD8+ cell isolations prepared using a post magnet apparatus (8E) or a ring magnet apparatus (8F). Contamination in the form of magnetic beads, red blood cells, and debris is indicated.

### DETAILED DESCRIPTION

Magnetic methods of separating WBCs from blood samples typically include mixing a sample containing blood cells (e.g., whole blood or a blood fraction) with magnetic particles conjugated to a binding member (e.g., an antibody) that specifically binds to WBCs or a subpopulation of WBCs (e.g., T cells, B cells, NK cells, monocytes, dendritic cells, granulocytes, or leukocytes), and separating the magnetic particle-bound cells from the sample using a magnetic field. Kits for performing magnetic separations are commercially available, e.g., from Dynal Biotech (Oslo, Norway), Miltenyi Biotech (Bergisch Gladbach, Germany), StemCell Technologies (British Columbia, Canada), BD Biosciences (Franklin Lakes, N.J.), Polysciences (Warrington, Pa.), and Bangs Laboratories (Fishcers, Ind.). Methods for magnetic separations are described in US 2007/0163963, which is incorporated herein by reference in its entirety.

Subpopulations or subsets of WBCs expressing specific cell surface proteins can be separated by utilization of magnetic particles conjugated to binding members specific for those proteins. Exemplary cell surface proteins include CD2, CD3, CD4, CD8, CD14, CD15, CD16, CD19, CD25, CD28, CD34, CD45, CD56, BMLF-1, LMP2, cytomegalovirus pp 65, Her-2/neu, MART-1, gp 100 (209-2M), and hTERT. Any cell surface marker CD1-CD227 can be used for this process by linking an antibody is against the marker of interest to a magnetic particle.

The new methods are improvements of the methods using such magnetic particles and can be used to isolate WBCs from any sample, e.g., tissue or fluid sample, that contains WBCs, preferably blood, e.g., whole blood. In some embodiments, the sample is whole blood, buffy coat, or a suspension of mononuclear cells (MNC). The sample can further include an anticoagulant, such as EDTA, heparin, citrate dextrose formula-A (ACD-A) or citrate phosphate dextrose (CPD). Whole blood can be from an animal (e.g., a mouse, rat, or rabbit) or from a human. Whole blood can be stored, e.g., with refrigeration, prior to separation. In other embodiments, the whole blood is used fresh, i.e., without storing.

Any blood fraction that contains WBCs can be used in the methods described herein. Preparation of blood fractions is well known in the art. For example, a buffy coat can be prepared by centrifuging a whole blood sample at about 200 g at room temperature and removing the band or layer that contains primarily leukocytes. Typically, this procedure yields a preparation with about 30 RBCs for each WBC. The use of gradients typically excludes the possibility of using automation. The complex process of visually extracting cells from a “fuzzy” interface between the variable gradients is difficult to perform without human input.

### Magnetic Apparatuses

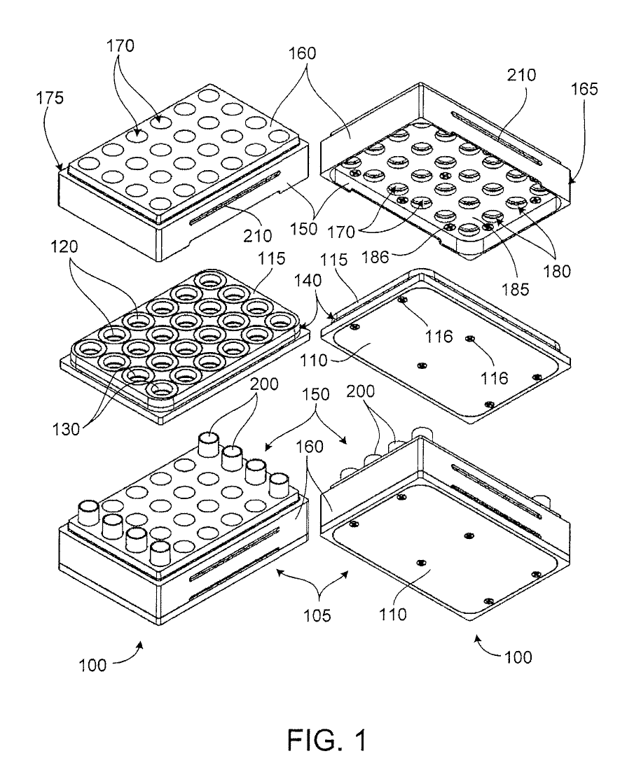

FIGS. 1-3 are schematic diagrams that show an exemplary magnetic apparatus 100. FIG. 1 shows an exploded view of the apparatus 100 as assembled. The apparatus is composed of a first portion 105 and a second portion 150. The first portion includes a solid support 110 upon which are fixedly attached a plurality of axially magnetized ring magnets 120 arranged in a rectangular array. Each ring magnet 120 defines an interior perimeter that defines an aperture 130 that is configured to receive the lower portion of a vial 200. The first portion also includes a housing 115 that surrounds that ring magnets and a tapered surface 140 that is configured to receive a corresponding surface 165 on the second portion 150 of the apparatus. The solid support 110 is fixedly attached to the housing 115 by means of one or more fasteners 116.

The second portion 150 includes a solid substrate 160 having surfaces 180 that define a plurality of tubular apertures 170 that are configured to receive the upper portion of a vial 200. The second portion 150 also includes a tapered surface 175 configured to receive a lid and a groove 210 configured to facilitate handling by a robotic arm. The second portion 150 also includes a bottom plate 185 fixedly attached to the solid substrate 160 by means of one or more fasteners 186.

The apparatus 100 can be assembled by joining the first portion 105 and the second portion 150 using the tapered surface 140 on the first portion 105 and the corresponding surface 165 on the second portion 150. When the first portion 105 and the second portion 150 are joined in this way, the apertures 130 defined by the interior perimeters of the ring magnets 120 line up with the tubular apertures 170 of the second portion 150 such that a vial 200 is contained within both the aperture 170 of the second portion 150 and the aperture 130 defined by the interior perimeter of the ring magnet 120. As a result, the lower portion of the vial 200 is exposed to the magnetic field produced by the ring magnet 120. The apparatus 100 can be disassembled by lifting the second portion 150 off of the first portion 105, thus removing the vials 200 from the magnetic field produced by the ring magnets 120.

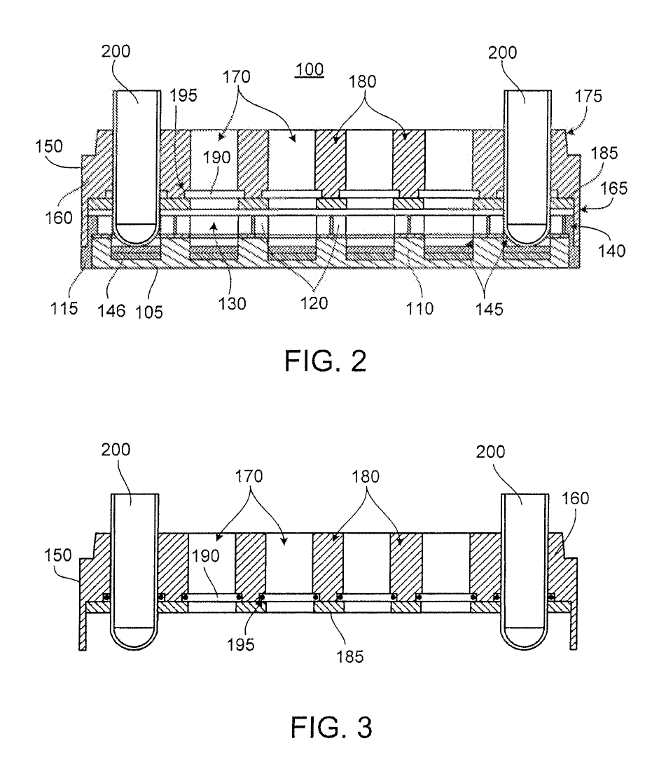

FIG. 2 is a cross-sectional view of an assembled apparatus 100. The assembled apparatus includes a first portion 105 that includes a solid support 110 upon which are fixedly attached a plurality of ring magnets 120, each of which defines an interior perimeter that defines an aperture 130 configured to receive the lower portion of a vial 200. The solid support 110 also includes an indentation 145 associated with each ring magnet 120. Each such indentation 145 defines an aperture contiguous with the aperture 130 defined by the ring magnet 120. The indentation 145 and aperture 130 defined by the ring magnet 120 are configured such that the bottom of a vial 200 placed within the apparatus 100 sits below the bottom plane of the ring magnets 120. The floor of each indentation 145 can include a cushion 146 configured to receive the bottom of the vial 200.

The first portion also includes a housing 115 with a tapered surface 140 that is configured to receive a corresponding surface 165 on the second portion 150 of the apparatus 100. The second portion 150 includes a solid substrate 160 having surfaces 180 that define a plurality of tubular apertures 170, each of which is configured to receive the upper portion of a vial 200. Each surface 180 defining a tubular aperture 170 includes a groove 195 configured to hold an o-ring 190. The o-rings 190 are further held in place by means of a bottom plate 185. The o-rings 190 hold the vials 200 in place when the second portion 150 is disassembled from the first portion 105. The o-rings 190 also allow for vials 200 to be inserted and removed from the second portion 150 of the apparatus 100. The second portion 150 also includes a tapered surface 175 configured to receive a lid.

FIG. 3 is a cross-sectional view of the second portion 150 of the apparatus, as seen when the vials 200 are not exposed to a magnetic field. As above, the second portion 150 includes a solid substrate 160 having surfaces 180 that define a plurality of tubular apertures 170 that are configured to receive the upper portion of a vial 200. Each surface 180 defining a tubular aperture 170 includes a groove 195 configured to hold an o-ring 190. The o-rings 190 hold the vials 200 in place as they are inserted and removed from the second portion 150.

Typically, the apparatus includes relatively strong magnets, e.g., strong rare earth magnets (e.g., neodymium-iron-boron permanent magnets). Strong electromagnets can also be suitable for performing magnetic separations. The magnetic field strength of the magnet (source) can be between about 20 and 500 kA/m or even greater (e.g., about 50, 100, 150, 200, 250, 300, 350, 400, 500, 600, 750, 1000, 10,000, or even 100,000 kA/m) in the vicinity of the magnetic particles. Typically, the magnetic field strength is at least about 300 kA/m in the vicinity of the magnetic particles. The magnets used for the methods can be ring magnets, e.g., a Ring Magnet 0.750″ OD×0.500″ ID×5 mm, Grade N50 (K&J Magnetics, Jamison, Pa.).

The cushions 146 can be used to prevent damage to the apparatus or pipette tip when the tip is inserted to the bottom of a vial 200. The cushion can be composed of any soft or flexible material, e.g., polyurethane, polyolefin foam rubber, felt, vinyl, or silicone foam rubber. An exemplary commercially-available cushion is available from McMaster-Carr, catalog number 8213K1.

FIG. 7 is an exemplary depiction of a unit cell of a set of axially magnetized ring magnets wherein, the polarity of the axial magnetization of each ring magnet (N or S) is inverted relative to its immediate neighbors. Each unit is a 2×2 array of magnets having a first pair of magnets forming a first diagonal of the array, and a second pair of magnets forming a second diagonal of the array, wherein the magnets from the first pair of magnets and the magnets from the second pair of magnets have opposite magnetic polarities.

### General Methodology

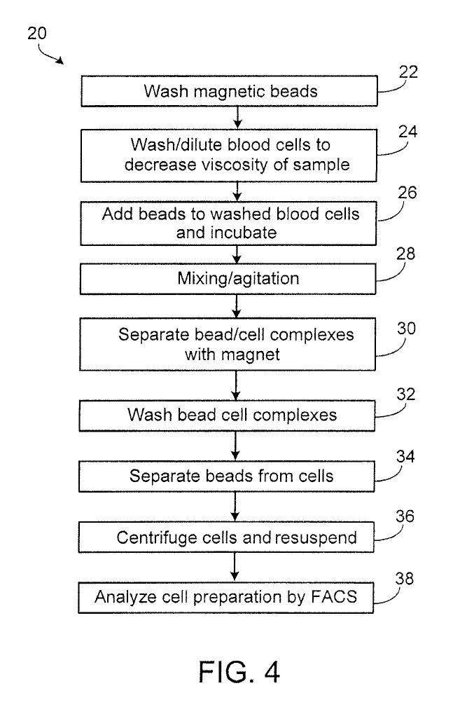

FIG. 4 is a flowchart that shows an exemplary set of steps 20 for cell preparation. These steps can be performed by an automated laboratory system. A sample that includes WBCs, e.g., whole blood, is optionally treated to reduce viscosity, e.g., by washing the cells one, two, three, or more times with an isotonic solution, such as Hank's Balanced Salts Solution (HBSS) with 0% to 5% serum, and also containing an anticoagulant, such as ACID citrate dextrose (ACD) solution (e.g., ACD) (step 24). Magnetic beads that bind to the desired WBC population, e.g., from Dynal Biotech (Oslo, Norway), are optionally washed (step 22) and added to the blood sample and the mixture is incubated for 10 minutes or more (e.g., 30 minutes, 45 minutes, 1 hour, or more) to allow the beads to bind to the WBCs (step 26). Optionally, the sample is agitated during bead binding to improve binding efficiency (step 28).

Following binding to the magnetic beads, the cells are separated from the sample (step 30) by subjecting sample containing the white blood cell/magnetic particle complexes to a magnetic field, e.g., a magnetic field of one or more of the ring magnets of an apparatus described herein. The unbound supernatant is removed, e.g., using a pipette or aspirator, and the magnetically attracted cells are washed up to four times with the isotonic solution (step 32). For each wash, an amount of isotonic solution equal to about one original sample volume or less is added to the magnetically bound cells, and the cells are allowed a period to re-attract to the magnet.

After the first wash step, this magnet re-attraction period should be at least 10 minutes to ensure binding of cells to the beads. For high purity (e.g., about 98%), nearly all of the supernatant can be removed from the tube after each wash and/or multiple washes can be used. For better yield with about 95% purity, it is useful to leave a small amount (about a tenth to a fifth of a sample volume) of supernatant in the tube following each wash. Thin-walled tubes can be used in all steps involving magnetic attraction.

An exemplary separation method includes placing the cells in a vial within an apparatus described herein with a first and second portion, such that the vials are held removably by the second portion of the apparatus; assembling the apparatus such that the vials are exposed to the magnetic field of the ring magnets and the magnetic particles are held within the vial by the magnetic field of the ring magnets; and removing the unbound supernatant. The magnetic particles can be subjected to one or more wash steps to remove residual unbound supernatant. The one or more wash steps can be performed by disassembling the apparatus such that the magnetic particles are no longer held within the vial by the magnetic field; adding an isotonic solution to the vial to re-suspend the magnetic particles within the solution; allowing time for the cells to bind to the magnetic particles; assembling the apparatus such that the vials are exposed to the magnetic field of the ring magnets and the magnetic particles are held within the vial by the magnetic field of the ring magnets; and removing the unbound solution. The apparatus can be assembled and disassembled by the automated laboratory system.

Following binding and washing, the cells and magnetic beads are separated (step 34), e.g., using a commercially available detachment solution (e.g., from Dynal) as described by the manufacturer. Optionally, the complexes can be agitated during detachment of the cells from the magnetic beads, and the cells can be centrifuged and resuspended (step 36). As discussed in further detail below, agitation is typically done using a vibration of at least about 200 or 400 to about 1000 rpm, e.g., at least about 700 or 800 rpm, and at an amplitude (e.g., a peak-to-peak amplitude) of about 1-20 mm (e.g., about 1-2, 1-5, 1-10, 2-5, 2-10, 2-20, 5-10, 1-3, 2-4, or 5-20 mm).

Optionally, cell yield is estimated by comparing the number of cells obtained, e.g., as measured by flow cytometry, to an estimate of the average number of the desired population of WBCs in the sample, e.g., human blood (e.g., 0.388×10⁶ cells/ml for CD8⁺ cells in human blood), or to a count of the number of the desired population of WBCs in the sample prior to processing (step 38).

Viability and purity of the isolated WBCs, such as CD8⁺ cells, are measured using standard techniques, e.g., flow cytometry. For example, 50 μl of a cell preparation can be mixed with 2000 of 5 μg/ml propidium iodide (PI) and FITC-conjugated antibody that binds specifically to the population of isolated WBCs. Dead cells can be identified by PI fluorescence. The desired WBCs will have fluorescence from FITC, whereas heterologous cells will not.

The apparatuses and methods disclosed herein can produce preparations of WBC preparations with 75% or greater cell yield (e.g., 80%, 85%, 90%, 95%, 98%, 99% or greater), 90% or greater cell viability (e.g., 92%, 95%, 98%, 99% or greater), and 80% or greater cell purity (e.g., 85%, 90%, 95%, 98%, 99% or greater).

Following isolation of WBCs, the cells can be “rested” before any downstream analysis by allowing the cells to remain undisturbed in cell culture medium or an isotonic solution for a period of about 2 hours to about 6 days, e.g., 2, 4, 6, 12, 15, 20, or 24 hours or 2, 3, 4, 5, or 6 days. This rest period can allow the cells to recover from any effects or damage the preparation method may have caused the cells.

Alternatively, the apparatuses and methods disclosed herein can be used for negative selection of WBCs, i.e., to remove WBCs or a population of WBCs from a sample that includes WBCs, e.g., whole blood. For negative selection of WBCs, the method is performed as above, a sample that includes WBCs, e.g., whole blood, is treated to reduce viscosity (e.g., by washing the cells one, two, three, or more times with an isotonic solution, such as Hank's Balanced Salts Solution (HBSS) with 0% to 5% serum, and also containing an anticoagulant, such as ACD or EDTA). Magnetic beads that bind to the WBC population one wants to remove, e.g., from Dynal Biotech (Oslo, Norway), are added and the mixture is incubated for 10 minutes or more (e.g., 30 minutes, 45 minutes, 1 hour, or more) to allow the beads to bind to the WBCs. Optionally, the mixture is agitated to improve efficiency of binding of the beads to the cells. Following binding to the magnetic beads, the cells are separated using a magnet device, e.g., an apparatus described herein. The unbound supernatant, which is substantially free (e.g., more than 75% free (more than 80%, 85%, 90%, 95%, 98%, or 99% free) of WBCs is removed and used for downstream processes. The methods described herein can be used for positive and/or negative selection of WBCs.

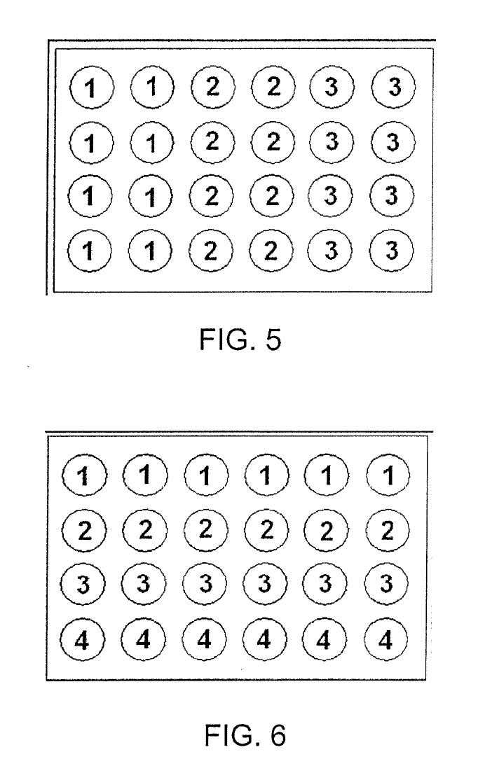

Typically, automated laboratory systems perform liquid manipulations using microplates with rectangular arrays of wells that hold liquids. A typical automated laboratory system uses a line of pipettors to add and remove liquid from the wells. A typical arrangement of wells in a 4 by 6 array is shown in FIG. 5. Typically, an automated laboratory system would use four pipettors used to manipulate such an array column by column. If three samples are allotted on the plate as shown in FIG. 5, with sample “1” allotted in the first two columns, sample “2” allotted in the second two columns, and sample “3” allotted in the last two columns, the leftmost columns are typically manipulated first and can experience longer resting periods between manipulations. This longer resting period can lead to variability of the treatment of individual samples and variability of final results. If the samples are, however, allotted each in an individual row as shown in FIG. 6, and the wells are manipulated column-by-column, the time between manipulations for the entire sample as a whole will be the same. By performing manipulations on a plate with such an arrangement, variability between the samples can be reduced. The samples allotted in rows can also be re-pooled following manipulations to reduce variability.

### High-Throughput Methods

The apparatuses and methods described herein can be used in high-throughput methods of WBC separations. For example, the procedures can be carried out in multi-well (e.g., 4-, 6-, 8-, 12-, 24-, 96-, or more-well) format for simultaneous processing of multiple samples. Furthermore, all of the steps can be adapted to be performed by a standard automated laboratory system robot.

Exemplary automated laboratory system robots include the Biomek® FX liquid handling system (Beckman-Coulter, Fullerton, Calif.), TekBench™ automated liquid handling platform (TekCel, Hopkinton, Mass.), and Freedom EVO® automation platform (Tecan Trading AG, Switzerland).

Open top tubes and/or plates are preferred for automated protocols, because of the difficulty of opening or closing vessels by automated systems. Mixing by agitation (e.g., agitation, shaking, or vibration) is advantageous for automated systems because the parameters can be set such that closing of tubes is not required, as it is for rocking or inversion mixing. Agitation/shaking modules are available for integration in automated systems and can be adapted for use in the protocols described herein.

By use of plates or racks adapted for larger volumes (e.g., with 4, 6, 8, 12, or 24 positions), the milliliter volumes typically used in blood cell preparations can be processed by automated platforms, either in one well of the plate or rack or in parallel in more than one. If the sample is split into more than one position, the final product can be pooled into one sample. Use of a 24-position rack with 2-ml cryogenic vials has been found to be suitable for blood cell purifications.

The apparatuses described herein are especially useful for automated high-throughput separation methods. For example, the robot can be programmed to fill the vials of the apparatus with the sample to be separated, to assemble the first and second portions of the apparatus to expose the sample to the magnetic field, and to disassemble the apparatus to remove the sample from the magnetic field.

### Implementation

The methods disclosed herein can be implemented in laboratory automation hardware controlled by a compatible software package (e.g., Biomek® FX software) programmed according to the new methods described herein or a new software package designed and implemented to carry out the specific method steps described herein. The methods can be implemented by computer programs using standard programming techniques following the method steps described herein.

The programs can be designed to execute on a programmable computer including at least one processor, at least one data storage system (including volatile and non-volatile memory and/or storage elements, e.g., RAM and ROM), at least one communications port that provides access for devices such as a computer keyboard, telephone, or a wireless, hand-held device, such as a PDA, and optionally at least one output device, such as a monitor, printer, or website. The central computer also includes a clock and a communications port that provides control of the lab automation hardware. These are all implemented using known techniques, software, and devices. The system also includes a database that includes data, e.g., data describing the procedure of one or more method steps described herein.

Program code is applied to data input by a user (e.g., location of samples to be processed, timing and frequency of manipulations, amounts of liquid dispensed or aspirated, transfer of samples from one location in the system to another) and data in the database, to perform the functions described herein. The system can also generate inquiries and provide messages to the user. The output information is applied to instruments, e.g., robots, that manipulate, heat, agitate, etc. the vessels that contain the blood samples as described herein. In addition, the system can include one or more output devices such as a telephone, printer, or a monitor, or a web page on a computer monitor with access to a website to provide to the user the results of testing (e.g., for purity, viability, and yield) of the blood samples.

Each program embodying the new methods is preferably implemented in a high level procedural or object-oriented programming language to communicate with a computer system. However, the programs can also be implemented in assembly or machine language if desired. In any case, the language can be a compiled or interpreted language.

Each such computer program is preferably stored on a storage medium or device (e.g., RAM, ROM, optical, magnetic) readable by a general or special purpose programmable computer, for configuring and operating the computer when the storage media or device is read by the computer to perform the procedures described herein. The system can also be considered to be implemented as a computer- or machine-readable storage medium (electronic apparatus readable medium), configured with a program, whereby the storage medium so configured causes a computer or machine to operate in a specific and predefined manner to perform the functions described herein.

The new methods can be implemented using various means of data storage. The files can be transferred physically on recordable media or electronically, e.g., by email on a dedicated intranet, or on the Internet. The files can be encrypted using standard encryption software from such companies as RSA Security (Bedford, Mass.) and Baltimore®. The files can be stored in various formats, e.g., spreadsheets or databases.

As used herein, the term “electronic apparatus” is intended to include any suitable computing or processing apparatus or other device configured or adapted for storing data or information. Examples of electronic apparatus suitable for use with the present invention include stand-alone computing apparatus; communications networks, including local area networks (LAN), wide area networks (WAN), Internet, Intranet, and Extranet; electronic appliances such as a personal digital assistants (PDAs), cellular telephones, pagers and the like; and local and distributed processing systems.

As used herein, “stored” refers to a process for encoding information on an electronic apparatus readable medium. Those skilled in the art can readily adopt any of the presently known methods for recording information on known media to generate manufactures comprising the sequence information.

A variety of software programs and formats can be used to store method data on an electronic apparatus readable medium. For example, the data and machine instructions can be incorporated in the system of the software provided with the automated system (e.g., the Biomek® FX software), represented in a word processing text file, formatted in commercially-available software such as WordPerfect® and MicroSoft®Word®, or represented in the form of an ASCII file, stored in a database application, such as Microsoft Access®, Microsoft SQL Server®, Sybase®, Oracle®, or the like, as well as in other forms. Any number of data processor structuring formats (e.g., text file or database) can be employed to obtain or create a medium having recorded thereon the relevant data and machine instructions to implement the methods described herein.

By providing information in electronic apparatus readable form, the programmable computer can communicate with and control the lab automation hardware to perform the methods described herein. One skilled in the art can input data in electronic apparatus readable form (or a form that is converted to electronic apparatus readable form) to describe the completion of various method steps by the lab automation hardware.

### EXAMPLE

### Example 1

### Exemplary Automated Blood Separation Protocol

An exemplary automated blood separation protocol includes the following steps:
Mix Blood & Beads
Settle on Magnet
Remove Supernatant
Wash 3-10 times
Add Media
Add Detachment Solution
Mix & Incubate
Settle on Magnet
Transfer Supernatant to Fresh Vials
Rinse Old Vials*
Settle on Magnet
Transfer Supernatant to Product Vials
Final Rinse
Settle on Magnet
Final Transfer

### Example 2

### Isolation of CD8+ Cells

CD8+ cells were isolated from whole blood using the apparatus depicted in FIGS. 1-3. Whole blood was obtained from five normal volunteers and transferred under sterile conditions from the anticoagulant tubes into 50 ml conical vials. The empty anticoagulant tubes were washed 3-5 times with ˜2 ml wash buffer (phosphate buffered saline (PBS), 0.1-2% fetal bovine serum (FBS), Pen/strp) and the wash buffer was added to the 50 ml conical vial containing the blood. The vials were then centrifuged for 10 minutes at ˜1800 rpm at between 10° C. and room temperature (RT). The supernatant was removed from each vial and discarded, 45 ml wash buffer was added to each vial, and the cells were resuspended by shaking. The vials were centrifuged again for 10 minutes at ˜1800 rpm at between 10° C. and RT, and the supernatant was removed and discarded. The vials containing the washed cells were placed on ice.

To prepare the magnetic beads, a 15 ml conical tube with Dynal/Invitrogen magnetic beads was placed on a rocker to mix. Twenty microliters of beads per milliliter of whole blood used were added to a 15 ml conical vial containing 7-10 ml of wash buffer. The vial was placed on a generic magnet for two minutes to attract the beads, and the supernatant was removed and discarded. The vial was removed from the magnet, and the beads were resuspended in the same original volume (20 μl/ml of whole blood) in wash buffer.

The washed beads were added to the vials containing the washed cells, mixed by pipetting and shaking, and allowed to stand for up to 1 hour. The vials containing beads and cells mixed and incubated similarly for 0-50 times. The sample containing beads and cells was then transferred to the vials of the magnetic apparatus by a robot, and the magnetic apparatus was assembled such that the bead-cell mixtures were exposed to the magnetic field generated by the magnets. The samples were incubated on the magnet for 1-4 minutes, and the supernatant was removed from each of the samples using the aspirator tip of a Biomek® FX robotic liquid handling system (Beckman-Coulter, Fullerton, Calif.). The magnetic apparatus was disassembled by the robot system, and the second portion of the apparatus was moved to an orbital shaker platform (BeckmanCoulter, Fullerton, Calif.). Two ml wash buffer was added to each of the vials, and the sample was mixed on the orbital shaker at 800 rpm for 1-10 minutes. The second portion of the apparatus was removed from the orbital shaker and reassembled with the first portion. The steps of incubation on the magnet, removal of the supernatant, disassembly of the apparatus, addition of wash buffer, shaking on the orbital shaker, and reassembly of the magnetic apparatus were repeated three times.

Following the final reassembly, the second portion of the apparatus was transferred to the orbital shaker, and 325 μl of detachment buffer (Hanks) were added to each vial. The samples were mixed by shaking for 1-60 minutes, and 54 μl of DETACHaBEAD™ CD4/CD8 (Dynal Biotech, Oslo, Norway) was added to each sample. The samples were mixed by shaking for 1-30 seconds and then incubated for 1-60 seconds, and the steps of mixing and incubation were repeated for 1-60 minutes.

The magnetic apparatus was reassembled to expose the samples to the magnetic field of the magnets. The supernatant from each vial containing CD8+ cells was transferred to the vials of a new second portion of the apparatus. The original vials on the magnet were washed once or twice with 200 μl of wash buffer, and each time the buffer was transferred to the corresponding vials of the new second portion of the apparatus. The magnetic apparatus was disassembled, and the new second portion of the apparatus was used to assemble the magnetic apparatus. The samples were incubated on the magnetic apparatus for 1-2 minutes, and the supernatant of each vial containing CD8 cells was transferred to a clean vial 200 microliters of wash buffer were added to the vials on the magnet, and the supernatant of each vial was transferred to the corresponding clean vial.

The clean vials were removed from the robot system, and the separate samples from each patient were pooled in 15 ml conical vials. Aliquots were removed for cell counting, and the remainder of the sample was centrifuged for 5 minutes at 1800 rpm at 10° C., the supernatant was removed, and the cells were resuspended in tissue culture medium (RPMI with 0.1-2% FCS) at 10⁶ cells/ml for analysis. Viability was assessed by flow cytometry using Propidium Iodide (PI) to stain of dead cells. Purity was assessed both by staining with CD8-FITC as well as staining the cells with CD235A, a marker for red blood cell contamination. The above data represents purity as analyzed by anti-CD8 FITC staining. Yield was calculated as the total yield of CD8 T cells/ml of blood. The results of the CD8+ cell isolation are shown in Table 1.

TABLE 1
Viability, purity, and yield of CD8+ cell isolation.

| | | | Yield |
|---|---|---|---|
| Blood # | Viability % | Purity % | 10⁵ cells/ml of blood |
| Sample #1 | 92 | 99 | 2.5 |
| Sample #2 | 96 | 99 | 6.0 |
| Sample #3 | 98 | 99 | 1.9 |
| Sample #4 | 99 | 98 | 1.8 |
| Sample #5 | 99 | 98 | 2.0 |

### Example 3

### Comparison of Ring and Post Magnets

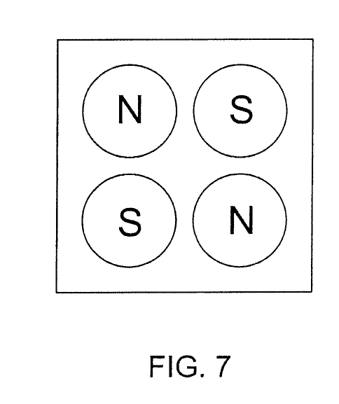

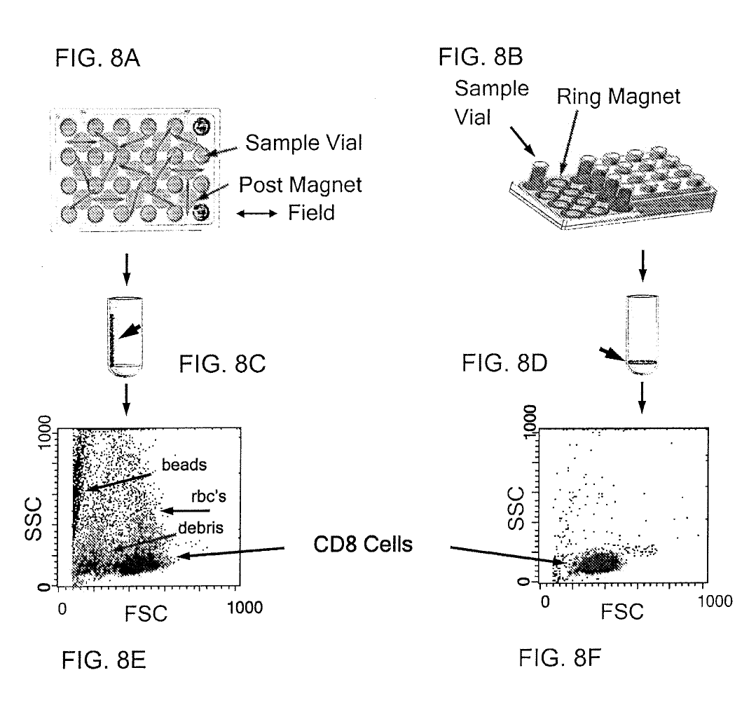

The properties of cells isolated with an apparatus containing ring magnets were compared with those isolated with an apparatus containing post magnets. CD8+ cell isolations were performed using an apparatus with post (cylindrical) magnets between sample positions (see FIG. 8A) or an apparatus with ring magnets as described herein (FIG. 8B). Each apparatus held 24 sample tubes, with magnets made of rare earth (Nd₂Fe₁₄B) material.

The post apparatus used 15 cylindrical magnets magnetized radially, which caused the magnetic beads to be pulled to an ‘extended’ line on the inside wall of the sample vial along the entire length of the magnet (see FIG. 8C). Being stretched over this extended length, all of the beads were not always exposed to wash buffer, potentially leaving bead-cell portions at risk for drying and possibly causing cell death and/or incomplete bead removal. Because the orientation of the post magnet fields was random, uneven distributions and edge effects from adjacent magnets may have been an issue.

In the apparatus with the ring magnets, there was one magnet per vial and bead-cell complex formed a smooth ring around the perimeter of the vial bottom (FIG. 8D). This bead-cell ring was easily bathed by the storage wash buffer. The axially magnetized ring magnets were mounted using alternate (+/−) pole polarity. CD8 cells isolated and collected using each apparatus essentially as described in Example 2. The resulting cell populations were tested for quality of separation using flow cytometry. 2-D scatter plots generated by cells from each isolation are shown for both post (FIG. 8E) and ring (FIG. 8F) configurations. In isolations performed using the post magnet apparatus, significant contamination was observed in the form of RBC, cell debris, and magnetic beads still present in the isolated fraction (FIG. 8E). The isolation using the ring magnet apparatus was significantly purer (FIG. 8F).

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. An apparatus for magnetically separating constituents of blood, the apparatus comprising:
a plurality of ring magnets, each ring magnet defining an interior perimeter enclosing an aperture;
a first portion having a solid support having the ring magnets fixedly attached thereon;
a second portion removably attached to the first portion, and
a solid substrate associated with the second portion, the solid substrate having surfaces defining a plurality of second apertures, each second aperture configured to removably hold a vial.

2. The apparatus of claim 1, wherein each of the plurality of ring magnets is axially magnetized.

3. The apparatus of claim 2, wherein the polarity of the axial magnetization of each ring magnet is inverted relative to its immediate neighbors.

4. The apparatus of claim 2, wherein the plurality of magnets comprises a repeating array of units, each unit being a 2×2 array of magnets having a first pair of magnets forming a first diagonal of the array, and a second pair of magnets forming a second diagonal of the array, wherein the magnets from the first pair of magnets and the magnets from the second pair of magnets have opposite magnetic polarities.

5. The apparatus of claim 1, wherein the solid support is composed of a non-magnetic material.

6. The apparatus of claim 1, wherein each of the plurality of ring magnets is configured to receive within its aperture a vial

7. The apparatus of claim 1, wherein the solid substrate comprises means for removably holding the vial.

8. A process for isolating magnetic particles to magnetically separate constituents of blood, the process comprising:
providing a vial containing a solution having magnetic particles;
placing the vial within a magnetic field created by a ring magnet, such that the particles are held within the vial by the magnetic field of the ring magnet; and
removing the solution from the vial, such that the magnetic particles remain isolated in the vial.

9. The process of claim 8, further comprising:
removing the vial from the magnetic field;
adding a second solution to the vial such that the magnetic particles are suspended within the second solution;
placing the vial within a magnetic field of a ring magnet; and
removing the solution from the vial.

10. A computer-readable medium with software encoded therein for causing a robot to isolate magnetic particles, the software including instructions for causing the robot to:
retrieve a vial containing a solution having magnetic particles;
place the vial within a magnetic field created by a ring magnet, such that the particles are held within the vial by the magnetic field created by the ring magnet; and
remove the solution from the vial, such that the magnetic particles remain isolated in the vial.

11. The computer-readable medium of claim 10, wherein the software further comprises instructions for causing a robot to:
remove the vial from the magnetic field;
add a second solution to the vial such that the magnetic particles are suspended within the second solution;
place the vial within the magnetic field created by the ring magnet, such that the particles are held within the vial by the magnetic field created by the ring magnet; and
remove the solution from the vial.

12. An apparatus for magnetically separating constituents of blood, the apparatus comprising:
a plurality of ring magnets, each ring magnet defining an interior perimeter enclosing an aperture; and
a first portion having a solid support having the ring magnets fixedly attached thereon.
